(19) United States
(12) Patent Application Publication
(10) Pub. No.: US 2011/0082335 A1
(43) Pub. Date: Apr. 7, 2011

(54) OPTICAL STRUCTURE OBSERVATION APPARATUS, STRUCTURE INFORMATION PROCESSING METHOD OF THE SAME AND ENDOSCOPE APPARATUS INCLUDING OPTICAL STRUCTURE OBSERVATION APPARATUS

(76) Inventors: Toshihiko Omori, Ashigarakami-gun (JP); Atsushi Ochiai, Kashiwa-shi (JP)

(21) Appl. No.: 12/896,068

(22) Filed: Oct. 1, 2010

(30) Foreign Application Priority Data
Oct. 2, 2009 (JP) 2009-230700

(51) Int. Cl. A61B 1/04; H04N 13/00

(52) U.S. Cl. 600/109; 348/45; 348/E13.021; 348/E07.085

(57) ABSTRACT

An optical structure observation apparatus which acquires a plurality of pieces of optical structure information of a measured object having a layer structure obtained by scanning a scan surface including a first direction which is a depth direction of the measured object and a second direction orthogonal to the first direction, using a low coherence light, while shifting a position along a third direction which is a direction orthogonal to the scan surface, and constructs an optical stereoscopic structure image based on the acquired plurality of pieces of the optical structure information, comprising:
a calculation region setting device which sets a plurality of calculation regions in the optical stereoscopic structure image;
a region characteristic information calculating device which performs prescribed processing on each calculation region, and calculates region characteristic information of the optical structure information;
a characteristic amount extracting device which extracts a characteristic amount in the calculation region based on the region characteristic information; and
a stereoscopic characteristic image generating device which generates a stereoscopic characteristic image based on the characteristic amount.

### BACKGROUND OF THE INVENTION

1. Field of the Invention

The present invention relates to an optical structure observation apparatus, a structure information processing method of the same and an endoscope apparatus including the optical structure observation apparatus, and particularly relates to an optical structure observation apparatus characterized in information processing on structure information of a measured object, a structure information processing method of the same and an endoscope apparatus including the optical structure observation apparatus.

2. Description of the Related Art

In recent years, as one of the methods for non-invasively obtaining the tomographic images of the insides of living bodies in, for example, a medical field or the like, optical coherence tomography (OCT) measurement has come to be used. As compared with ultrasound measurement, the OCT measurement has the advantage that the resolution is about 10 μm, namely, one digit higher, and the detailed tomographic images of the insides of living bodies can be obtained.

The OCT measurement is a method for acquiring an optical tomographic image of a specified region, as described above. Under endoscope, for example, a cancer lesion site is identified by observation using a normal illumination light endoscope and a special light endoscope. The region is subjected to the OCT measurement, thereby allowing distinguishing how far the cancer lesion site is invaded. The optical axis of the measuring light is two-dimensionally scanned, thereby allowing three-dimensional information to be acquired in combination with depth information by the OCT measurement.

An amalgam of the OCT measurement and a three-dimensional computer graphic technology enables a three-dimensional structure model to be displayed with micrometer-order resolution. Accordingly, the three-dimensional structure model by the OCT measurement is hereinafter referred to as an optical stereoscopic structure image (or optical stereoscopic structure information).

Typically, in an endoscopic diagnosis of colon cancer, crypts are observed under endoscope. Classification is made according to mucosal structures of the large intestine, which are referred to as pit patterns. In order to observe such crypts structure in detail, characteristics of the tissue structure are made distinct using a dye or the like, and observation is made by means of a magnifying endoscope. In order to solve a problem of increase in test time and cost owing to distribution of the dye and to allow easy observation, a method of reproducing color contrast by image processing on pits and projections of a two-dimensional mucosal surface (Japanese Patent Application Laid-Open No. 2001-25025) has been proposed.

On the other hand, three-dimensional measurement of a large intestine mucosa by means of the aforementioned OCT measurement can extract the three dimensional structure of a crypt structure, and acquire a structure analogous to the pit pattern as information around the surface. Further, since the OCT measurement acquires a three-dimensional structure, variation in depth direction of a crypt structure is observed. It is known that, in case of canceration of the large intestine, the crypt structure of a mucosal layer is deteriorated. It is also known that extraction of the crypts therefrom and analysis of the three-dimensional shape allows a difference between a normal site and a lesion site to be numerically analyzed (Journal of Biomedical Optics Vol. 13, p. 054055 (2008)).

However, the pit pattern diagnosis is a method of determining the progress strictly based on an image of a mucosal surface. Information relating to the depth of invasion is only empirical. Although the crypt structure on a surface can be determined, the internal structure cannot be determined. Further, in extraction of crypts by three-dimensional measurement using the OCT measurement, it is visually difficult to identify the structure. It is also difficult to image a stereoscopic variation of a lesion site.

### SUMMARY OF THE INVENTION

The present invention is made in view of these situations. It is an object of the present invention to provide an optical structure observation apparatus capable of three-dimensionally analyzing the internal structure of a measured object having a layer structure such as living tissue, a structure information processing method of the same, and an endoscope apparatus including the optical structure observation apparatus.

To attain the object, an optical structure observation apparatus according to an aspect of the present invention is an optical structure observation apparatus which acquires a plurality of pieces of optical structure information of a measured object having a layer structure obtained by scanning a scan surface including a first direction which is a depth direction of the measured object and a second direction orthogonal to the first direction, using a low coherence light, while shifting a position along a third direction which is a direction orthogonal to the scan surface, and constructs an optical stereoscopic structure image based on the acquired plurality of pieces of the optical structure information, including: a calculation region setting device which sets a plurality of calculation regions in the optical stereoscopic structure image; a region characteristic information calculating device which performs prescribed processing on each calculation region, and calculates region characteristic information of the optical structure information; a characteristic amount extracting device which extracts a characteristic amount in the calculation region based on the region characteristic information; and a stereoscopic characteristic image generating device which generates a stereoscopic characteristic image based on the characteristic amount.

In the optical structure observation apparatus of the first aspect of the present invention, the calculation region setting device sets the plurality of calculation regions in the optical stereoscopic structure image, the region characteristic information calculating device performs prescribed processing on each calculation region and calculates region characteristic information of the optical structure information, the characteristic amount extracting device extracts the characteristic amount in the calculation region based on the region characteristic information, and the stereoscopic characteristic image generating device generates the stereoscopic characteristic image based on the characteristic amount, thereby enabling the internal structure of a measured object having a layer structure such as living tissue to be three-dimensionally analyzed.

As with the optical structure observation apparatus according to a second aspect of the present invention, in the optical structure observation apparatus set forth in the first aspect, the calculation region setting device preferably includes: a middle layer extracting device which extracts a desired middle layer in the measured object from the optical structure information configuring the optical stereoscopic structure image; a layer flattening device which flattens the middle layer; a structure image converting device which reconstructs the optical stereoscopic structure image with the flattened middle layer as a reference layer, and generates a three-dimensional converted optical structure image; and a parallel region setting device which sections the three-dimensional converted optical structure image by a parallel plane parallel to the reference layer on the three-dimensional converted optical structure image, and sets a plurality of parallel regions orthogonal to the reference layer at prescribed intervals, the parallel regions being set as the calculation regions.

As with the optical structure observation apparatus according to a third aspect of the present invention, in the optical structure observation apparatus set forth in the second aspect, the characteristic amount extracting device preferably includes: a parallel tomographic image generating device which generates a parallel tomographic image based on the region characteristic information in each parallel region; and an image analyzing device which extracts the characteristic amount by performing a spatial frequency analysis on the parallel tomographic image.

As with the optical structure observation apparatus according to a fourth aspect of the present invention, in the optical structure observation apparatus set forth in the second or third aspect, the region characteristic information extracting device preferably performs any one of integration processing, maximum intensity projection processing, and minimum intensity projection processing on the optical structure information in the parallel region along a direction orthogonal to the reference layer as the prescribed processing, and thereby extracts the region characteristic information.

As with the optical structure observation apparatus according to a fifth aspect of the present invention, in the optical structure observation apparatus set forth in any one of the second to fourth aspects, the stereoscopic characteristic image is preferably a distribution image of the characteristic amount.

As with the optical structure observation apparatus according to a sixth aspect of the present invention, it is preferable that the optical structure observation apparatus set forth in any one of the second to fifth aspects further include a display control device which causes a display device to display at least any one of the optical stereoscopic image, the three-dimensional converted optical structure image and the stereoscopic characteristic image.

As with the optical structure observation apparatus according to a seventh aspect of the present invention, in the optical structure observation apparatus set forth in any one of the first to sixth aspects, the measured object is preferably a living luminal organ.

As with the optical structure observation apparatus according to an eighth aspect of the present invention, in the optical structure observation apparatus set forth in the seventh aspect, the reference layer is preferably muscularis mucosae.

As with the optical structure observation apparatus according to a ninth aspect of the present invention, in the optical structure observation apparatus set forth in the seventh or eighth aspect, the optical structure information is preferably structure information including crypt structure in the living luminal organ.

As with the optical structure observation apparatus according to a tenth aspect of the present invention, in the optical structure observation apparatus set forth in any one of the seventh to ninth aspects, the characteristic amount preferably represents a degree of canceration of the living luminal organ based on the optical structure information.

A structure information processing method for an optical structure observation apparatus according to an eleventh aspect of the present invention which acquires a plurality of pieces of optical structure information of a measured object having a layer structure obtained by scanning a scan surface including a first direction which is a depth direction of the measured object and a second direction orthogonal to the first direction, using a low coherence light, while shifting a position along a third direction which is a direction orthogonal to the scan surface, and constructs an optical stereoscopic structure image based on the acquired plurality of pieces of the optical structure information, includes: a calculation region setting step which sets a plurality of calculation regions in the optical stereoscopic structure image; a region characteristic information calculating step which performs prescribed processing on each calculation region, and calculates region characteristic information of the optical structure information; a characteristic amount extracting step which extracts a characteristic amount in the calculation region based on the region characteristic information; and a stereoscopic characteristic image generating step which generates a stereoscopic characteristic image based on the characteristic amount.

An endoscope apparatus according to a twelfth aspect of the present invention, includes: the optical structure observation apparatus according to any one of the first to tenth aspects; and an endoscope which includes an insertion section to be inserted into a lumen, wherein a probe emitting and receiving the low coherence light is capable of being inserted into the insertion section.

As described above, the present invention exerts an advantageous effect capable of three-dimensionally analyzing the internal structure of a measured object having a layer structure such as living tissue.

### BRIEF DESCRIPTION OF THE DRAWINGS

FIG. 1 is an outside view showing a diagnostic imaging apparatus according to the present invention;

FIG. 2 is a block diagram showing an internal configuration of an OCT processor of FIG. 1;

FIG. 3 is a sectional view of an OCT probe of FIG. 2;

FIG. 4 is a view showing a state of obtaining optical structure information using the OCT probe guided out of a forceps port of an endoscope of FIG. 1;

FIG. 5 is a block diagram showing a configuration of a processing section of FIG. 2;

FIG. 6 is a flowchart illustrating an operation of the diagnostic imaging apparatus of FIG. 1;

FIG. 7 is a first diagram for illustrating processing of FIG. 6;

FIG. 8 is a view showing an optical stereoscopic structure image generated by an optical stereoscopic structure image constructing section of FIG. 5;

FIG. 9 is a second diagram for illustrating the processing of FIG. 6;

FIG. 10 is a third diagram for illustrating the processing of FIG. 6;

FIG. 11 is a view showing an optical stereoscopic structure image converted by an optical stereoscopic structure image converting section of FIG. 5;

FIG. 12 is a fourth diagram for illustrating the processing of FIG. 6;

FIG. 13 is a view showing one example of a parallel tomographic image where a pit pattern generated by a parallel tomographic image generating section of FIG. 5 is represented;

FIG. 14 is a fifth diagram for illustrating the processing of FIG. 6;

FIG. 15 is a view showing one example where a stereoscopic characteristic image generated by the processing in FIG. 6 is displayed;

FIG. 16 is a view showing an arbitrary tomographic image in a depth direction of the stereoscopic characteristic image in FIG. 15; and

FIG. 17 is a view showing a parallel tomographic image of the stereoscopic characteristic image of FIG. 15 at an arbitrary depth position.

### DETAILED DESCRIPTION OF THE PREFERRED EMBODIMENTS

Hereinafter, a best mode for carrying out the present invention will be described.

<Appearance of Diagnostic Imaging Apparatus>

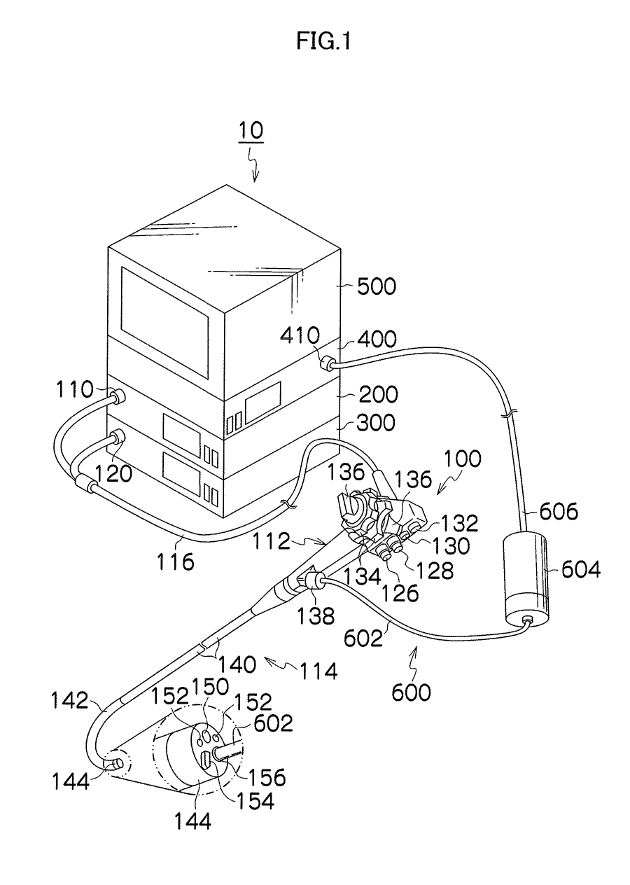

FIG. 1 is an outside view showing a diagnostic imaging apparatus according to the present invention.

As shown in FIG. 1, a diagnostic imaging apparatus 10 as an optical structure observation apparatus of this embodiment mainly includes an endoscope 100, an endoscope processor 200, a light source device 300, an OCT processor 400 and a monitor device 500 as a display device. The endoscope processor 200 may be configured to contain the light source device 300.

The endoscope 100 includes a hand operation section 112, and an insertion section 114 provided to connect to the hand operation section 112. An operator grips and operates the hand operation section 112, inserts the insertion section 114 into a body of a subject, and thereby performs observation.

The hand operation section 112 is provided with a forceps insertion section 138, and the forceps insertion section 138 is caused to communicate with a forceps port 156 of a distal end portion 144. In the diagnostic imaging apparatus 10 according to the present invention, by inserting an OCT probe 600 from the forceps insertion section 138, the OCT probe 600 is guided out of the forceps port 156. The OCT probe 600 includes an insertion section 602 which is inserted from the forceps insertion section 138 and is guided out of the forceps port 156, an operation section 604 for an operator to operate the OCT probe 600, and a cable 606 which is connected to the OCT processor 400 via a connector 410.

<Configuration of Endoscope, Endoscope Processor and Light Source Device>

[Endoscope]

An observation optical system 150, an illumination optical system 152 and a CCD (not illustrated) are placed at the distal end portion 144 of the endoscope 100.

The observation optical system 150 forms an image of a subject on a light receiving surface of the CCD, which is not illustrated. The CCD converts the subject image formed on the light receiving surface into an electrical signal by each light receiving element. The CCD of this embodiment is a color CCD in which color filters of three primary colors of red (R), green (G) and blue (B) are placed in predetermined arrangement (Bayer arrangement, honeycomb arrangement) in each pixel.

[Light Source Device]

The light source device 300 causes visible light to be incident on a light guide, which is not illustrated. One end of the light guide is connected to the light source device 300 via an LG connector 120, and the other end of the light guide is opposed to the illumination optical system 152. The light emitted from the light source device 300 is emitted from the illumination optical system 152 via the light guide and illuminates the visual field range of the observation optical system 150.

[Endoscope Processor]

To the endoscope processor 200, an image signal outputted from the CCD is inputted through an electric connector 110. The analog image signal is converted into a digital image signal in the endoscope processor 200, and is subjected to processing necessary for being displayed on the screen of the monitor device 500.

The data of the observation image obtained in the endoscope 100 is outputted to the endoscope processor 200, and the image is displayed on the monitor device 500 which is connected to the endoscope processor 200.

<Internal Configurations of OCT Processor and OCT Probe>

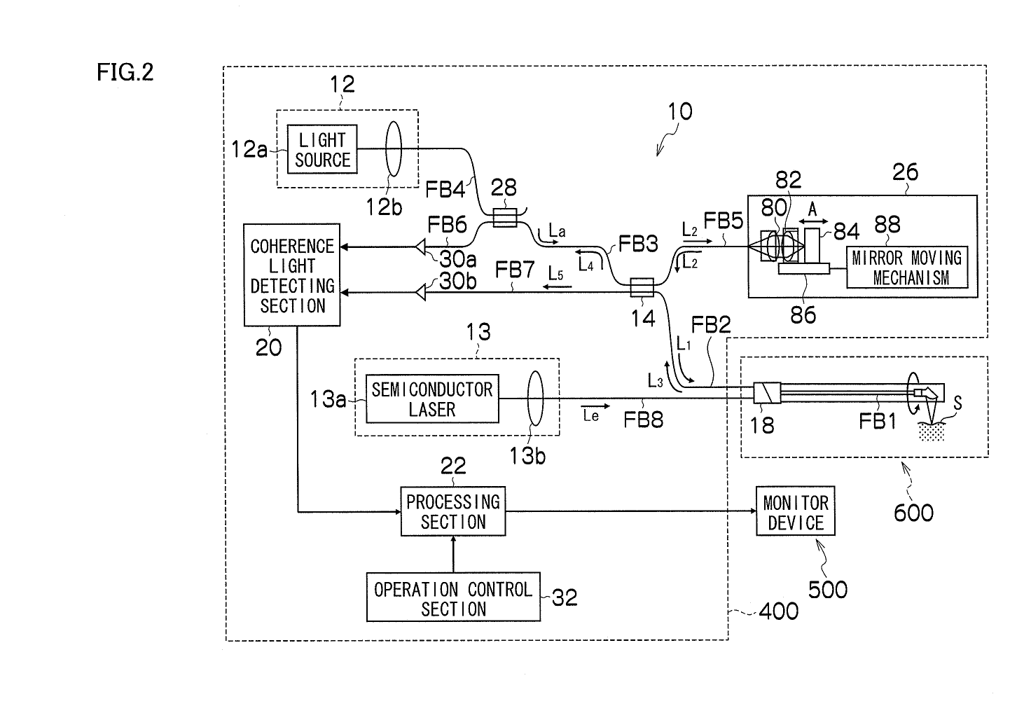

FIG. 2 is a block diagram showing the internal configuration of the OCT processor of FIG. 1.

[OCT Processor]

The OCT processor 400 and the OCT probe 600 shown in FIG. 2 are for acquiring the optical tomographic image of a measured object by an optical coherence tomography (OCT: Optical Coherence Tomography) measurement method, and includes: a first light source (first light source unit) 12 which emits a light La for measurement; an optical fiber coupler (branching/multiplexing section) 14 which branches the light La emitted from the first light source 12 into a measuring light (first luminous flux) L1 and a reference light L2, and generates a coherence light L4 by multiplexing a return light L3 from a measurement object S which is a subject and the reference light L2; the OCT probe 600 including a rotation side optical fiber FB1 which guides the measuring light L1 branched by the optical fiber coupler 14 to the measurement subject, and guides the return light L3 from the measurement object; a fixed side optical fiber FB2 which guides the measuring light L1 to the rotation side optical fiber FB1 and guides the return light L3 guided by the rotation side optical fiber FB1; an optical connector 18 which connects the rotation side optical fiber FB1 rotatably with respect to the fixed side optical fiber FB2, and transmits the measuring light L1 and the return light L3; a coherence light detecting section 20 which detects the coherence light L4 generated in the optical fiber coupler 14 as a coherence signal; and a processing section 22 which processes the coherence signal detected by the coherence light detecting section 20 to acquire optical structure information. Further, an image is displayed on the monitor device 500 based on the optical structure information acquired in the processing section 22.

Further, the OCT processor 400 includes a second light source (second light source unit) 13 which emits an aiming light (second luminous flux) Le for indicating a mark of measurement, an optical path length adjusting section 26 which adjusts an optical path length of the reference light L2, an optical fiber coupler 28 which separates the light La emitted from the first light source 12, detectors 30a and 30b which detect return lights L4 and L5 multiplexed in the optical fiber coupler 14, and an operation control section 32 which inputs various conditions to the processing section 22, and makes change of setting and the like.

In the OCT processor 400 shown in FIG. 2, as the optical paths for guiding and transmitting various lights including the aforementioned emission light La, aiming light Le, measuring light L1, reference light L2, return light L3 and the like to and from the components such as each of optical devices, various optical fibers FB (FB3, FB4, FB5, FB6, FB7, FB8 and the like) including the rotation side optical fiber FB1 and the fixed side optical fiber FB2 are used.

The first light source 12 emits light (for example, laser light or low coherence light of a wavelength of 1.3 μm) for measurement of OCT. The first light source 12 emits for example laser light La centered at 1.3 μm, which is in the infrared region, while sweeping frequencies at a certain period. The first light source 12 includes a light source 12a which emits laser light or low coherence light La, and a lens 12b which condenses the light La emitted from the light source 12a. Though described in detail later, the light La emitted from the first light source 12 is divided into the measuring light L1 and the reference light L2 in the optical fiber coupler 14 through the optical fibers FB4 and FB3, and the measuring light L1 is inputted into the optical connector 18.

Further, the second light source 13 emits a visible light as the aiming light Le in order to make the observation site easily confirmable. For example, a red semiconductor laser light of a wavelength of 0.66 μm, a He—Ne laser light of a wavelength of 0.63 μm, a blue semiconductor laser light of a wavelength of 0.405 μm and the like can be used. Thus, the second light source 13 includes a semiconductor laser 13a which emits a laser light of, for example, red, blue or green, and a lens 13b which condenses the aiming light Le emitted from the semiconductor laser 13a. The aiming light Le emitted from the second light source 13 is inputted to the optical connector 18 through the optical fiber FB8.

In the optical connector 18, the measuring light L1 and the aiming light Le are multiplexed, and are guided to the rotation side optical fiber FB1 in the OCT probe 600.

The optical fiber coupler (branching/multiplexing section) 14 is made of an optical fiber coupler of, for example, 2 by 2, and is optically connected to the fixed side optical fiber FB2, the optical fiber FB3, the optical fiber FB5 and the optical fiber FB7 respectively.

The optical fiber coupler 14 splits the light La incident from the first light source 12 through the optical fibers FB4 and FB3 into the measuring light (first luminous flux) L1 and the reference light L2, causes the measuring light L1 to be incident on the fixed side optical fiber FB2, and causes the reference light L2 to be incident on the optical fiber FB5.

Further, the optical fiber coupler 14 multiplexes the light L2 which is incident on the optical fiber FB5, is subjected to the frequency shift and change of optical path length by the optical path length adjusting section 26 which will be described later and returns through the optical fiber FB5, and the light L3 which is acquired by the OCT probe 600 which will be described later and is guided from the fixed side optical fiber FB2, and emits the multiplexed light to the optical fiber FB3 (FB6) and the optical fiber FB7.

The OCT probe 600 is connected to the fixed side optical fiber FB2 via the optical connector 18. The measuring light L1 multiplexed with the aiming light Le is incident on the rotation side optical fiber FB1 from the fixed side optical fiber FB2 through the optical connector 18. The incident measuring light L1 multiplexed with the aiming light Le is transmitted by the rotation side optical fiber FB1 to irradiate the measurement object S. The return light L3 from the measurement object S is acquired. The acquired return light L3 is transmitted by the rotation side optical fiber FB1, and is emitted to the fixed side optical fiber FB2 through the optical connector 18.

The optical connector 18 multiplexes the measuring light (first luminous flux) L1 and the aiming light (second luminous flex) Le.

The coherence light detecting section 20 is connected to the optical fiber FB6 and the optical fiber FB7, and detects the coherence lights L4 and L5 generated by multiplexing the reference light L2 and the return light L3 by the optical fiber coupler 14 as coherence signals.

Here, the OCT processor 400 has the detector 30a which is provided on the optical fiber FB6 branched from the optical fiber coupler 28 and detects the light intensity of the coherence light L4, and the detector 30b which detects the light intensity of the coherence light L5 on the optical path of the optical fiber FB7.

The coherence light detecting section 20 Fourier transforms the coherence light L4 detected from the optical fiber FB6, and the coherence light L5 detected from the optical fiber FB7 based on detection results from the detectors 30a and 30b, and thereby detects the intensity of the reflection light (or backscattering light) at each depth position of the measurement object S.

The processing section 22 detects the region where the OCT probe 600 and the measurement object S are in contact in the measurement position, more accurately, the region where the surface of the probe outer barrel (which will be described later) of the OCT probe 600 and the surface of the measurement object S are regarded to be in contact with each other from the coherence signal extracted in the coherence light detecting section 20, further acquires the optical structure information from the coherence signal detected in the coherence light detecting section 20, generates the optical stereoscopic structure image based on the acquired optical structure information, and outputs the image produced by applying various processes to the optical stereoscopic structure image to the monitor device 500. The detailed configuration of the processing section 22 will be described later.

The optical path length adjusting section 26 is disposed at the emission side (more specifically, the end portion of the optical fiber FB5 at the side opposite to the optical fiber coupler 14) of the reference light L2 of the optical fiber FB5.

The optical path length adjusting section 26 includes a first optical lens 80 which collimates the light emitted from the optical fiber FB5 into collimated light, a second optical lens 82 which condenses the a light collimated by the first optical lens 80, a reflective mirror 84 which reflects the light condensed by the second optical lens 82, a base 86 which supports the second optical lens 82 and the reflective mirror 84, and a mirror moving mechanism 88 which moves the base 86 in the direction parallel with the optical axis direction, and adjusts the optical path length of the reference light L2 by changing the distance between the first optical lens 80 and the second optical lens 82.

The first optical lens 80 collimates the reference light L2 emitted from the core of the optical fiber FB5 into collimated light, and condenses the reference light L2 reflected by the reflective mirror 84 into the core of the optical fiber FB5.

Further, the second optical lens 82 condenses the reference light L2 collimated by the first optical lens 80 on the reflective mirror 84, and collimates the reference light L2 reflected by the reflective mirror 84 into collimated light. In this manner, a confocal optical system is formed by the first optical lens 80 and the second optical lens 82.

Further, the reflective mirror 84 is disposed at the focal point of the light condensed by the second optical lens 82, and reflects the reference light L2 condensed by the second optical lens 82.

Thereby, the reference light L2 emitted from the optical fiber FB5 is made into collimated light by the first optical lens 80, and is condensed on the reflective mirror 84 by the second optical lens 82. Thereafter, the reference light L2 reflected by the reflective mirror 84 is made into collimated light by the second optical lens 82, and is condensed into the core of the optical fiber FB5 by the first optical lens 80.

Further, the base 86 fixes the second optical lens 82 and the reflective mirror 84. The mirror moving mechanism 88 moves the base 86 in the direction of the optical axis of the first optical lens 80 (direction of arrows A in FIG. 2).

By moving the base 86 in the direction of arrows A with the mirror moving mechanism 88, the distance between the first optical lens 80 and the second optical lens 82 can be changed, and the optical path length of the reference light L2 can be adjusted.

The operation control section 32 as an extracted region setting device includes an input device such as a keyboard and a mouse, and a control device which manages various conditions based on the inputted information, and is connected to the processing section 22. The operation control section 32 performs input, setting, change and the like of various processing conditions and the like in the processing section 22 based on the instruction of the operator inputted from the input device.

The operation control section 32 may cause the monitor device 500 to display an operation screen, or may be additionally provided with a display section and cause the display section to display the operation screen. Further, in the operation control section 32, the operation control of the first light source 12, the second light source 13, the optical connector 18, the coherence light detecting section 20, the optical path length and the detectors 30a and 30b and setting of various conditions may be performed.

[OCT Probe]

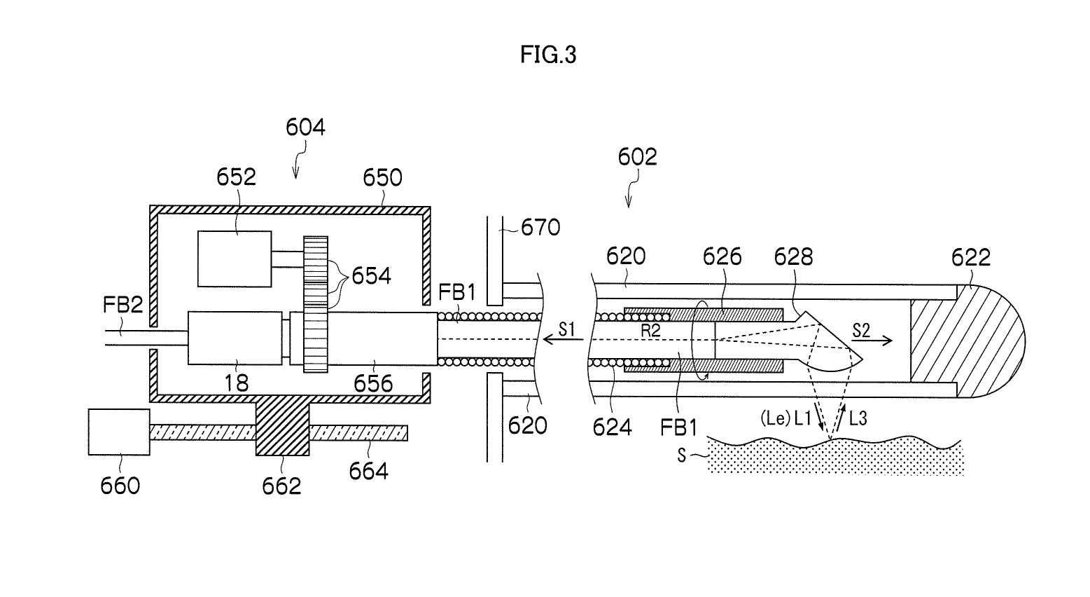

FIG. 3 is a sectional view of the OCT probe of FIG. 2.

As shown in FIG. 3, a distal end portion of the insertion section 602 includes a probe outer barrel 620, a cap 622, the rotation side optical fiber FB1, a spring 624, a fixing member 626 and an optical lens 628.

The probe outer barrel (sheath) 620 is a cylindrical member having flexibility, and is made of a material through which the measuring light L1 multiplexed with the aiming light Le in the optical connector 18 and the return light L3 transmit. In the probe outer barrel 620, a part of the distal end (distal end of the rotation side optical fiber FB1 at the side opposite to the optical connector 18, hereinafter, referred to as the distal end of the probe outer barrel 620) side where the measuring light L1 (aiming light Le) and the return light L3 pass only has to be formed of a material (transparent material) which transmits the light over the entire periphery, and the portions other than the distal end may be formed of a material which does not transmit a light.

The cap 622 is provided at the distal end of the probe outer barrel 620, and closes the distal end of the probe outer barrel 620.

The rotation side optical fiber FB1 is a linear member, housed in the probe outer barrel 620 along the probe outer barrel 620, guides the measuring light L1, which has been emitted from the fixed side optical fiber FB2 and multiplexed with the aiming light Le emitted from the optical fiber FB8 in the optical connector 18, to the optical lens 628, guides the return light L3, which has been acquired from the measurement object S via the optical lens 628 after irradiating the measurement object S with the measuring light L1 (aiming light Le), to the optical connector 18, and causes the return light L3 to be incident on the fixed side optical fiber FB2.

Here, the rotation side optical fiber FB1 and the fixed side optical fiber FB2 are connected by the optical connector 18, and are optically connected in the state in which rotation of the rotation side optical fiber FB1 is not transmitted to the fixed side optical fiber FB2. Further, the rotation side optical fiber FB1 is disposed in the state rotatable with respect to the probe outer barrel 620 and movable in the axial direction of the probe outer barrel 620.

The spring 624 is fixed to the outer periphery of the rotation side optical fiber FB1. Further, the rotation side optical fiber FB1 and the spring 624 are connected to the optical connector 18.

The optical lens 628 is disposed at the distal end (distal end of the rotation side optical fiber FB1 at the side opposite to the optical connector 18) at the measuring side of the rotation side optical fiber FB1, and the distal end portion is formed into a substantially spherical shape for condensing the measuring light L1 (aiming light Le) emitted from the rotation side optical fiber FB1 onto the measurement object S.

The optical lens 628 irradiates the measurement object S with the measuring light L1 (aiming light Le) emitted from the rotation side optical fiber FB1, condenses the return light L3 from the measurement object S, and causes the return light L3 to be incident on the rotation side optical fiber FB1.

The fixing member 626 is disposed on the outer peripheries of the connecting portions of the rotation side optical fiber FB1 and the optical lens 628, and fixes the optical lens 628 to the end portion of the rotation side optical fiber FB1. Here, the method of fixing the rotation side optical fiber FB1 and the optical lens 628 by the fixing member 626 is not specially limited. The fixing member 626, and the rotation side optical fiber FB1 and the optical lens 628 may be bonded and fixed by an adhesive, or may be fixed with a mechanical structure using a bolt and the like. As the fixing member 626, any member such as a zirconia ferrule and a metal ferrule may be used as long as it is used for fixing, holding or protecting the optical fiber.

Further, the rotation side optical fiber FB1 and the spring 624 are connected to a rotary barrel 656 which will be described later. The optical lens 628 is rotated in the direction of the arrow R2 with respect to the probe outer barrel 620 by rotating the rotation side optical fiber FB1 and the spring 624 by the rotary barrel 656. Further, the optical connector 18 includes a rotary encoder, and detects the irradiating position of the measuring light L1 from the positional information (angle information) of the optical lens 628 based on the signal from the rotary encoder. More specifically, the angle is detected with respect to the reference position in the rotational direction of the rotating optical lens 628, thereby detecting the measuring position.

Further, the rotation side optical fiber FB1, the spring 624, the fixing member 626 and the optical lens 628 are configured to be movable in the direction of the arrow S1 (forceps port direction) and the direction of S2 (direction of the distal end of the probe outer barrel 620) inside the probe outer barrel 620 by a drive section which will be described later.

Further, the left side of FIG. 3 is a view showing the outline of the drive section for the rotation side optical fiber FB1 and the like in the operation section 604 of the OCT probe 600.

The probe outer barrel 620 is fixed to a fixing member 670. In contrast with this, the rotation side optical fiber FB1 and the spring 624 are connected to the rotary barrel 656, and the rotary barrel 656 is configured to rotate through a gear 654 in accordance with the rotation of a motor 652. The rotary barrel 656 is connected to the optical connector 18, and the measuring light L1 and the return light L3 are transmitted between the rotation side optical fiber FB1 and the fixed side optical fiber FB2 through the optical connector 18.

Further, a frame 650 containing these elements includes a support member 662, and the support member 662 has a screw hole, which is not illustrated. A ball screw 664 for advance and retreat movement is engaged in the screw hole, and a motor 660 is connected to the ball screw 664 for advance and retreat movement. Accordingly, by rotationally driving the motor 660, the frame 650 is moved to advance and retreat, and thereby, the rotation side optical fiber FB1, the spring 624, the fixing member 626, and the optical lens 628 can be moved in the directions of S1 and S2 in FIG. 3.

The OCT probe 600 is configured as above, and the rotation side optical fiber FB1 and the spring 624 are rotated in the direction of the arrow R2 in FIG. 3 by the optical connector 18, whereby the OCT probe 600 irradiates the measurement object S with the measuring light L1 (aiming light Le) emitted from the optical lens 628 while scanning in the direction of the arrow R2 (the circumferential direction of the probe outer barrel 620), and acquires the return light L3. The measurement object S is irradiated with the aiming light Le as a spot light of, for example, a blue color, red color or green color, and the reflection light of the aiming light Le is displayed on the observation image displayed on the monitor device 500 as a bright spot.

Thereby, in the entire periphery in the circumferential direction of the probe outer barrel 620, a desired site of the measurement object S can be accurately captured, and the return light L3 reflected at the measurement object S can be acquired.

Further, when a plurality of pieces of optical structure information for generating an optical stereoscopic structure image is to be acquired, the optical lens 628 is moved to the terminal end of the movable range in the direction of the arrow S1 by the drive section, moves in the direction S2 by a predetermined amount while acquiring the optical structure information including a tomographic image, or moves to the terminal end in the movable range while alternately repeating optical structure information acquisition and movement by a predetermined amount in the direction S2.

Like this, a plurality of pieces of optical structure information in a desired range are obtained for the measurement object S, and based on a plurality of pieces of optical structure information, an optical stereoscopic structure image can be obtained.

More specifically, the optical structure information in the depth direction (first direction) of the measurement object S is obtained by the coherence signal, and scanning is performed in the direction of the arrow R2 (circumferential direction of the probe outer barrel 620) of FIG. 3 for the measurement object S, whereby, the optical structure information on the scan surface including the first direction and the second direction orthogonal to the first direction can be acquired, and by further moving the scan surface along the third direction orthogonal to the scan surface, a plurality of pieces of optical structure information for generating the optical stereoscopic structure image can be acquired.

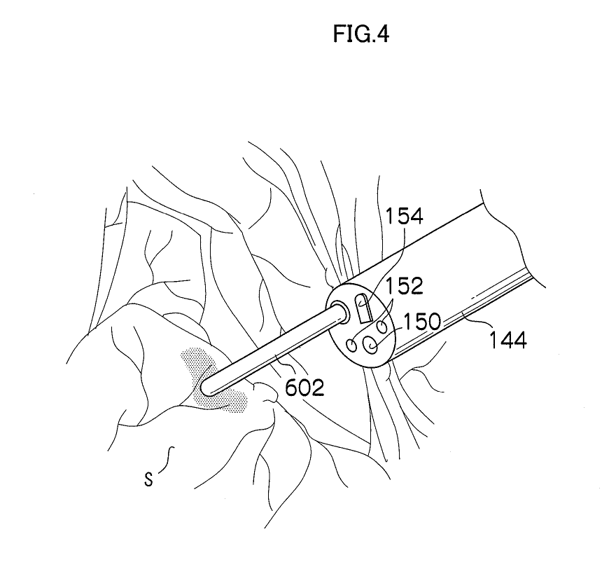

FIG. 4 is a view showing the state in which optical structure information is obtained by using the OCT probe guided out of the forceps port of the endoscope of FIG. 1. As shown in FIG. 4, the distal end portion of the insertion section 602 of the OCT probe is moved close to a desired site of the measurement object S, and optical structure information is obtained. When a plurality of pieces of optical structure information in a desired range is to be acquired, the OCT probe 600 main body does not have to be moved, but the optical lens 628 only has to be moved in the probe outer barrel 620 by the aforementioned drive section.

[Processing Section]

FIG. 5 is a block diagram showing a configuration of the processing section of FIG. 2.

As shown in FIG. 5, the processing section 22 of the OCT processor 400 includes an optical structure information detecting section 220, an optical stereoscopic structure image constructing section 221, a middle layer extracting section 222 as a middle layer extracting device, a flattening processing section 223 as a layer flattening device, an optical stereoscopic structure image converting section 224 as a structure image converting device, a parallel region setting section 225 as a parallel region setting device, a region characteristic information calculating section 226 as a region characteristic information calculating device, a parallel tomographic image generating section 227 as a parallel tomographic image generating device, a canceration level discriminating section 228 (parallel tomographic image analyzing section) as an image analyzing device, a stereoscopic characteristic image generating section 229 as a stereoscopic characteristic image generating device, a display control section 230 and an I/F (interface) section 231.

A calculation region setting device includes the middle layer extracting section 222, the flattening processing section 223, the optical stereoscopic structure image converting section 224 and the parallel region setting section 225.

A characteristic amount extracting device includes the parallel tomographic image generating section 227 and the canceration level discriminating section 228.

The optical structure information detecting section 220 detects optical structure information from the coherence signal detected in the coherence light detecting section 20. Further, the optical stereoscopic structure image constructing section 221 generates an optical stereoscopic structure image based on the optical structure information detected by the optical structure information detecting section 220.

The middle layer extracting section 222 extracts muscularis mucosae as a middle layer when the measurement object S is the mucosa of a large intestine, for example. When the mucosal epithelium is a squamous epithelium as in an esophagus, the middle layer extracting section 222 extracts a basement membrane (basal layer) as a middle layer.

The middle layer can be set by a setting signal of the operation control section 32 via the I/F section 231.

The flattening processing section 223 shifts the data in the depth direction so that the extracted muscularis mucosae is in a certain reference position in order to flatten the layer position of the muscularis mucosae extracted by the middle layer extracting section 222. The flattening processing section 223 may be configured by a processing section that fits the position of the muscularis mucosae to an arbitrary function from the two-dimensional optical structure information or the three-dimensional optical structure information.

The optical stereoscopic structure image converting section 224 converts an optical stereoscopic structure image so that muscularis mucosae become the reference surface of the optical stereoscopic structure image.

The reference surface is not limited to muscularis mucosae, and may be a mucosal surface, a basal layer (when the mucosal epithelium is an original squamous epithelium). However, in the case of a large intestine, it is more preferable that the muscularis mucosae be set as the reference surface.

The parallel region setting section 225 sections the optical stereoscopic structure image converting section 224 in different depths by parallel planes along a direction orthogonal to the reference layer, and sets a plurality of parallel regions whose number of divisions has been set by the setting signal from the operation control section 32 via the I/F section 231.

The number of divisions may be arbitrarily set. For example, the operator confirms the optical stereoscopic structure image converting section 224 on the monitor device 500, and is thereby capable of setting the number of divisions on the parallel region setting section 225 via the IF section 231 in consideration of the observation site (e.g., the large intestine), the disease state and the like.

The region characteristic information calculating section 226 integrates the optical structure information in each parallel region along the direction orthogonal to the reference layer, and calculates a pit pattern as region characteristic information in each parallel region.

The parallel tomographic image generating section 227 generates the parallel tomographic image, which is an integrated image where the pit pattern as the region characteristic information calculated by the region characteristic information calculating section 226 is represented.

The parallel tomographic image generated by the parallel tomographic image generating section 227 is not limited to an integrated image, but may be any one of an MIP (Maximum intensity projection) image and an MINIP (Minimum intensity projection) image. For example, the operator can set the processing method in the parallel tomographic image generating section 227 by the setting signal of the operation control section 32 via the I/F section 231. It is preferable that the processing of the parallel tomographic image generating section 227 be used where appropriate so as to allow the characteristics of the structure to be seen by being highlighted. Further, in the structure of regular arrangement such as a crypt structure of a large intestine normal site, it is preferable that the image generated by the processing in the parallel tomographic image generating section 227 be an integrated image. Further, the parallel tomographic image generated by the parallel tomographic image generating section 227 may be a tomographic image of an extracted region without performing processing such as integration processing, maximum intensity projection processing and minimum intensity projection processing.

The canceration level discriminating section 228 performs a spatial frequency analysis (Fourier analysis) on the parallel tomographic image generated by the parallel tomographic image generating section 227, and performs an image analysis (canceration level discrimination) for discriminating between a normal site and an abnormal site in the respective parallel tomographic images at the different height positions. After finishing the parallel tomographic image analysis (canceration level discrimination) at a certain height position, the canceration level discriminating section 228 performs an analogous analysis (canceration level discrimination) on the parallel tomographic image at a different height.

Analysis regions of the parallel tomographic images for discrimination between a normal site and an abnormal site (canceration level discrimination) can be set by a setting signal of the operation control section 32 via the IN section 231.

The processing (canceration level discrimination) for detecting abnormality of the pattern arrangement in the canceration level discriminating section 228 is not limited to the Fourier transformation. Instead, other pattern recognition methods may be adopted. Further, the discrimination is not limited to the discrimination from the patterns of the plurality of pits. Instead, the shape of a pit is extracted, and the discrimination may be made according to a deviation from a round. The discrimination may be made according to a distance to the adjacent pit. The discrimination parameter may be calculated by combining these factors in the mixed manner.

The stereoscopic characteristic image generating section 229 generates a stereoscopic characteristic image where the optical stereoscopic structure image constructed by the optical stereoscopic structure image constructing section 221 has further been reconstructed, based on information acquired from the analysis result in the canceration level discriminating section 228.

The display control section 230 caused the monitor device 500 to display at least one of the optical stereoscopic image, a three-dimensional converted optical structure image, the stereoscopic characteristic image and the like, based on an instruction signal from the operation control section 32.

The I/F section 231 is a communication interface section which transmits a setting signal and an instruction signal from the operation control section 32 to each of the sections.

For example, in order to cause the stereoscopic characteristic image generating section 229 to display the lesion site emphasizing the three-dimensional regions, the display control section 230 applies a certain signal to the optical stereoscopic structure image constructed by the optical stereoscopic structure image constructing section 221 at the lesion site and reconstructs the stereoscopic characteristic image on the monitor device 500, and emphases are placed with gradations and colors different from those of the normal site. The reconstructed stereoscopic characteristic image allows observation on stereoscopic variation in the lesion site, more specifically, the three-dimensional stereoscopic distribution image of the lesion site.

The display control section 230 is capable of causing the monitor device 500 to display any tomographic image of the stereoscopic characteristic image reconstructed by the setting signal of the operation control section 32 via the I/F section 231. Any of the tomographic images is displayed two-dimensionally, thereby enabling two-dimensional distribution images of a lesion site at any part (depth) to be observed in detail.

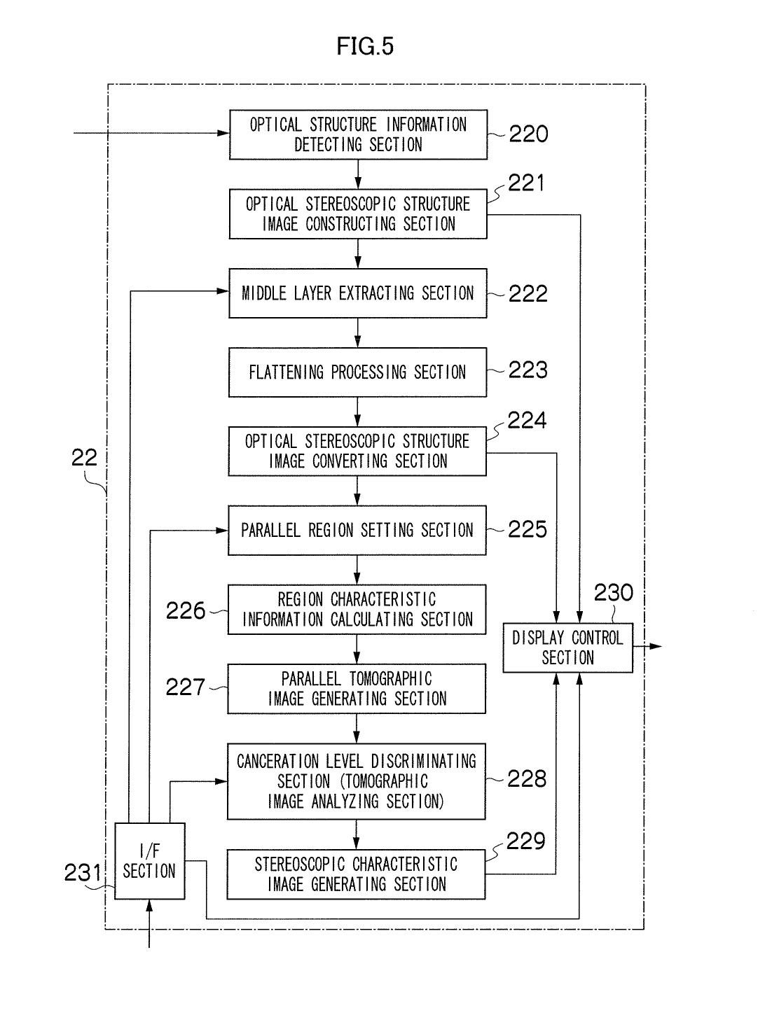

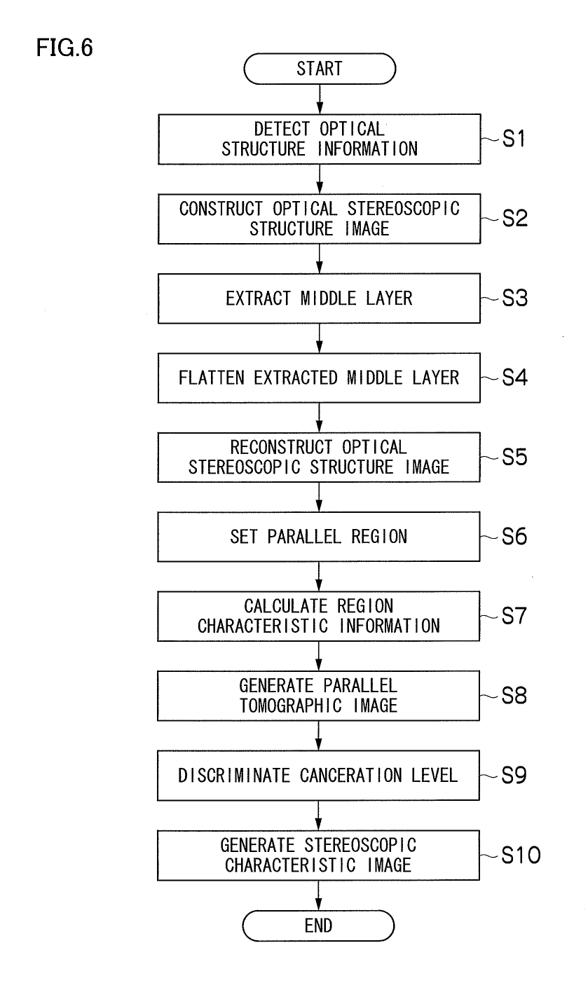

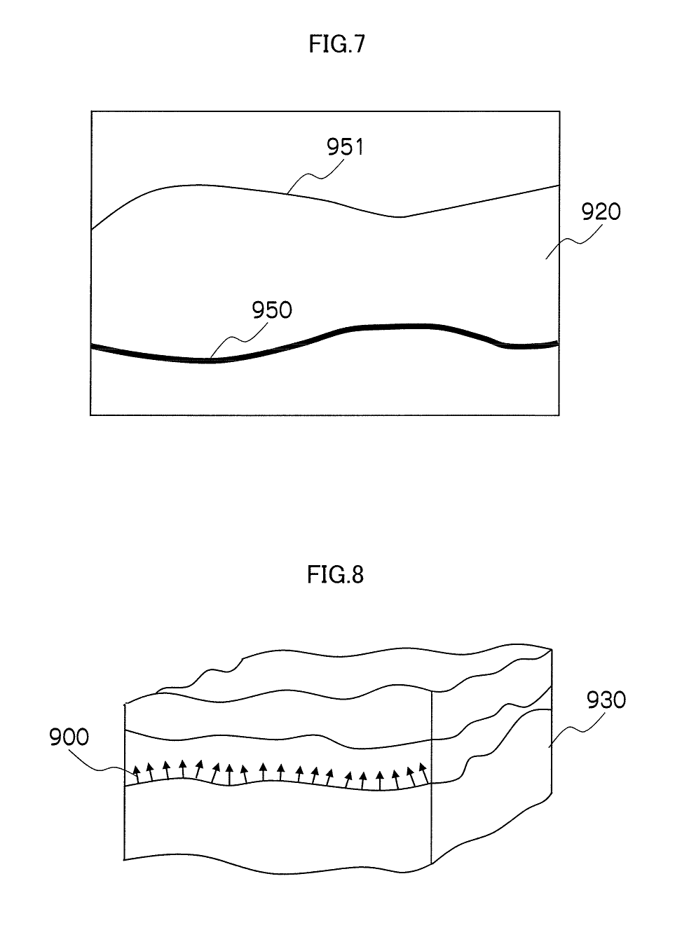

An operation of the diagnostic imaging apparatus 10 as the optical structure observation apparatus of this embodiment thus configured will be described using a flowchart of FIG. 6 with reference to FIGS. 7 to 16.

FIG. 6 is the flowchart illustrating the operation of the diagnostic imaging apparatus of FIG. 1. FIGS. 7 to 17 are diagrams for illustrating the processing of FIG. 6.

An operator turns on power of each of the sections of the endoscope 100, the endoscope processor 200, the light source device 300, the OCT processor 400 and the monitor device 500, which configure the diagnostic imaging apparatus 10, brings the distal end portion of the insertion section 602 of the OCT probe 600 guided out of the forceps port of the endoscope 100 close to the mucosa of a large intestine (measurement object S), for example, and starts optical scanning by the OCT probe 600.

In processing section 22 of the OCT processor 400 of the diagnostic imaging apparatus 10, as shown in FIG. 6, the optical structure information detecting section 220 detects the optical structure information on a scan surface 920 for configuring a tomographic image as shown in FIG. 7 from the coherence signal detected by the coherence light detecting section 20 (step S1), and the optical stereoscopic structure image constructing section 221 generates an optical stereoscopic structure image 930 as shown in FIG. 8 based on the optical structure information detected by the optical structure information detecting section 220 (step S2).

In the optical stereoscopic structure image 930 of FIG. 8, crypts are formed substantially vertically on the mucosal layer with muscularis mucosae 950 as a basal plate, and therefore, the orientation of the crypts (the arrows 900 in FIG. 8) are random orientations.

At this time, the display control section 230 can output the image of the optical stereoscopic structure image 930 from the optical stereoscopic structure image constructing section 221 to the monitor device 500 by the instruction signal of the operation control section 32 via the I/F section 231.

Next, in the processing section 22, the display control section 230 causes the monitor device 500 to display the optical structure information on the scan surface 920 for configuring the optical stereoscopic structure image 930 generated by the optical stereoscopic structure image constructing section 221, and the middle layer extracting section 222 extracts muscularis mucosae 950 as a middle layer in the optical structure information on the scan surface 920 when the measurement object S is the mucosa of a large intestine, for example (step S3).

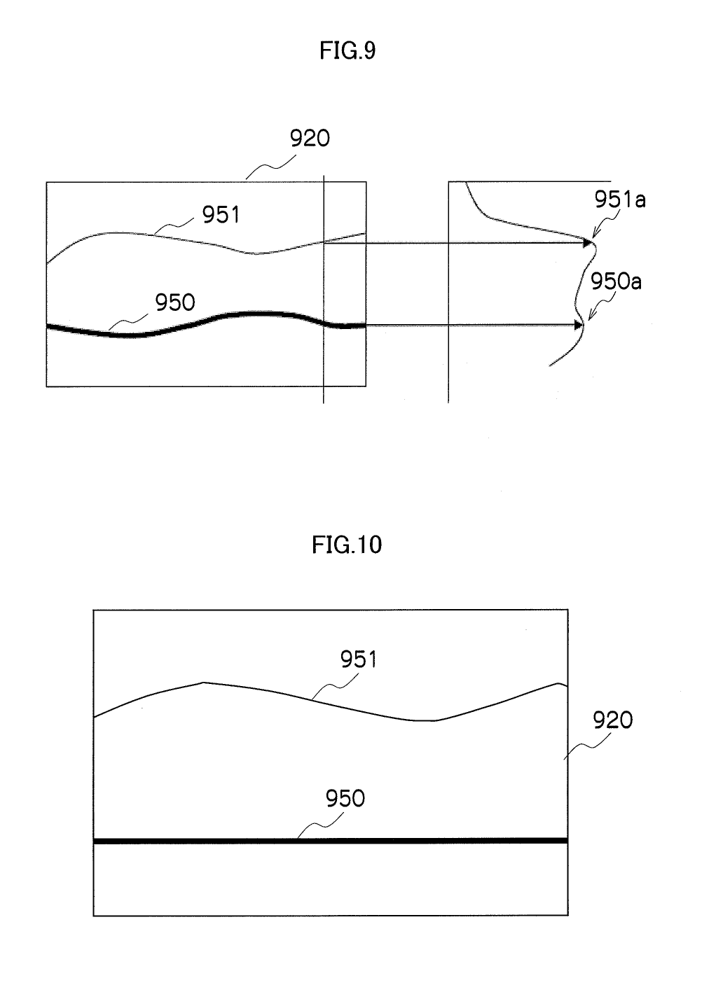

More specifically, the middle layer extracting section 222 extracts muscularis mucosae by analyzing the intensity of an image signal. That is, the middle layer extracting section 222 determines that in each scan surface 920 for configuring the optical stereoscopic structure image 930, a portion 951a with strong image signal intensity is a mucosal surface 951, and a next portion 950a with strong image signal intensity corresponds to the muscularis mucosae 950, and extracts the layer position of the muscularis mucosae 950, as shown in FIG. 9.

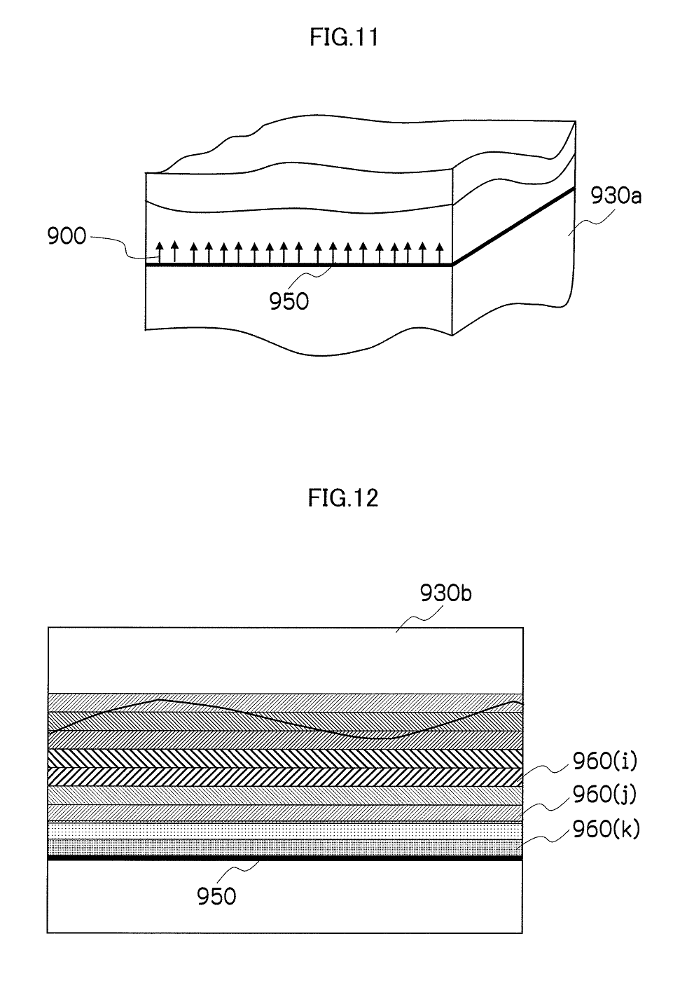

In the processing section 22, the flattening processing section 223 then shifts the optical structure information in the depth direction so as to locate the extracted muscularis mucosae 950 in a certain reference position in order to flatten the layer position of the muscularis mucosae 950 extracted by the middle layer extracting section 222, and flattens the muscularis mucosae 950 as shown in FIG. 10 (step S4). Subsequently, in the processing section 22, the stereoscopic structure image converting section 224 converts the optical stereoscopic structure image 930 into an optical stereoscopic structure image 930a as shown in FIG. 11 so that the muscularis mucosae 950 flattened by the flattening processing section 223 becomes the reference surface of the optical stereoscopic structure image (step S5).

At this time, the display control section 230 can output the image of the optical stereoscopic structure image 930a from the optical stereoscopic structure image converting section 224 to the monitor device 500 by the instruction signal of the operation control section 32 via the I/F section 231. The crypts are formed substantially vertically in the mucosal layer with the flattened muscularis mucosae 950 as the basal plate in the optical stereoscopic structure image 930a as shown in FIG. 11. Therefore, the orientations (arrow 900 in FIG. 11) of the crypts are regular orientations when the crypts are normal. In this manner, the state of the crypts can be easily determined visually by means of the optical stereoscopic structure image 930a.

Next, in the processing section 22, as shown in FIG. 12, the parallel region setting section 225 sections the optical stereoscopic structure image converting section 224 in different depths by parallel planes along the direction orthogonal to the reference layer, and sets the plurality of parallel regions whose number of divisions has been set by the setting signal from the operation control section 32 via the I/F section 231 (step S6). FIG. 12 shows a section 930b of the stereoscopic characteristic image of FIG. 11 in the depth direction.

At this time, the number of divisions of the plurality of the parallel regions in the parallel region setting section 225 may arbitrarily be set. For example, the operator verifies the optical stereoscopic structure image converting section 224 on the monitor device 500, and is thereby able to set the number of divisions of the parallel regions on the parallel region setting section 225 via the I/F section 231 in consideration of the observation site (e.g., the large intestine), the disease state and the like.

FIG. 12 shows eight-part division as an example. Hereinafter, for the sake of simplicity of description, the parallel regions 960 (i), 960 (j) and 960 (k) at three different depths are used.

In the processing section 22, the region characteristic information calculating section 226 integrates the optical structure information of each parallel region along the direction orthogonal to the basal layer, and calculates the pit pattern as the region characteristic information of each parallel region (step S7).

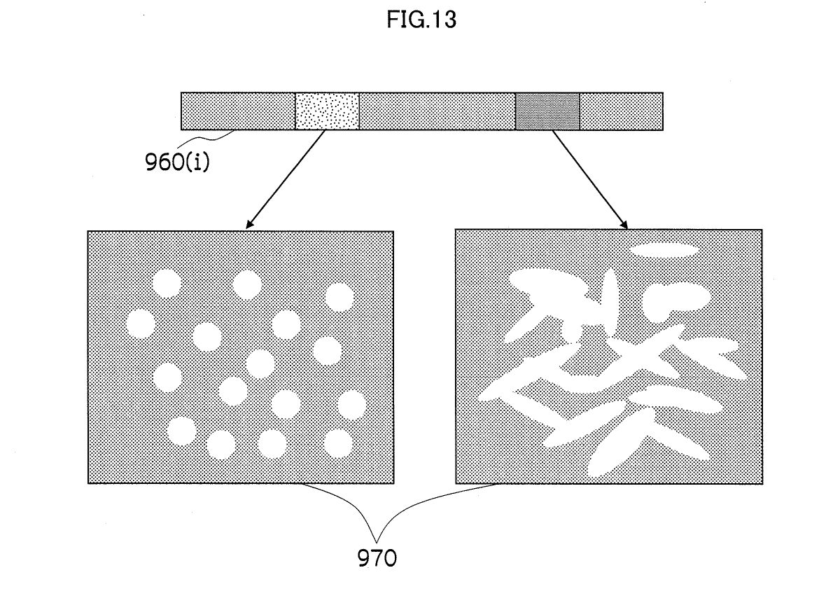

Subsequently, in the processing section 22, as shown in FIG. 13, the parallel tomographic image generating section 227 generates a parallel tomographic image 970, which is an integrated image where a pit pattern emerges as the region characteristic information in the parallel region 960 (i), for example, and has been calculated at the region characteristic information calculating section 226, for each parallel region (step S8).

Next, in the processing section 22, the canceration level discriminating section 228 performs the spatial frequency analysis (Fourier analysis) on the parallel tomographic image 970 generated by the parallel tomographic image generating section 227, and performs the image analysis (canceration level discrimination) for discriminating between a normal site and an abnormal site in the respective parallel tomographic images in the parallel regions 960 (i), 960 (j) and 960 (k) at the different height positions (step S9).

For example, in the pit pattern of a normal site of the large intestine, circular patterns having the diameter of about 100 μm are regularly arranged. The canceration level discriminating section 228 sets, for example, an analysis region of about 500 μm centered at a certain point in the parallel tomographic image based on the setting signal of the operation control section 32 via the I/F section 231, and performs the Fourier transformation on the analysis region. With respect to the image after the Fourier transformation, in a case where the analysis region is a normal area, a sharp peak is detected around a period of 100 μm. On the other hand, in a case where the analysis region is a lesion site, the regular arrangement disappears. Accordingly, in the image after the Fourier transformation, the peak becomes dull, and finally disappears.

The canceration level discriminating section 228 extracts a converted value (amount of peak appearance) of the Fourier analysis image as a characteristic amount.

The canceration level discriminating section 228 repeatedly performs the Fourier analysis while moving on the parallel tomographic image in the analysis region, and thereby acquires the canceration information of the crypt structure based on the characteristic amount (amount of peak appearance) and discriminates between a normal site and an abnormal site of the crypt structure (or a degree of canceration).

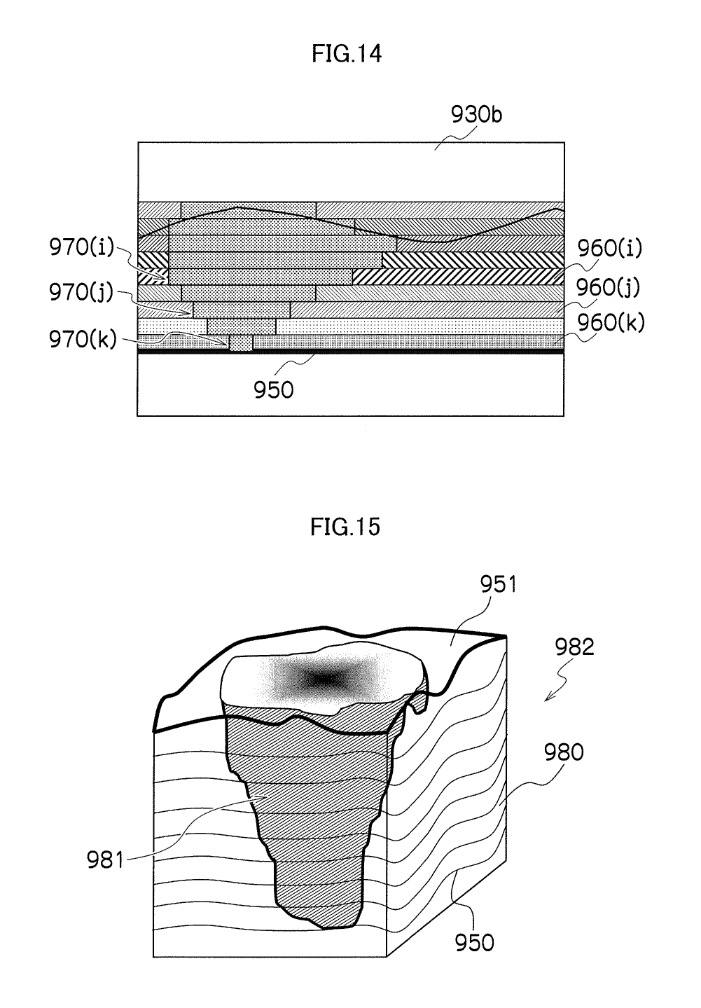

In step S9, after completion of the analysis on the parallel tomographic image at a certain height, the canceration level discriminating section 228 subsequently performs an analogous analysis on the parallel tomographic image at a different height. Accordingly, the canceration level discriminating section 228 acquires not only information in the horizontal direction of the lesion site, but also canceration information in the depth direction. More specifically, as shown in FIG. 14, the canceration level discriminating section 228 is capable of acquiring an area where abnormally shaped crypts are dominant in the parallel tomographic images at different depths (parallel regions 960 (i), 960 (j) and 960 (k)), which is the three-dimensional distribution of the canceration information of areas 971 (i), 971 (j) and 971 (k).

Next, in the processing section 22, the stereoscopic characteristic image generating section 229 generates a stereoscopic characteristic image 981 as a distribution image of the canceration information, which for example has been reconstructed by superimposing the canceration information image 981 on the optical stereoscopic structure image 980 constructed by the optical stereoscopic structure image constructing section 221, based on the information acquired from the analysis result of the canceration level discriminating section 228, as shown in FIG. 15 (step S10).

According to this embodiment, in the processing section 22, the display control section 230 causes the monitor device 500 to display at least any one of the optical stereoscopic structure image 980 by the optical stereoscopic structure image constructing section 221, the three-dimensional converted optical structure image 930 (see FIG. 8) by the optical stereoscopic structure image converting section 224, and the stereoscopic characteristic image 982 by the stereoscopic characteristic image generating section 229, responsive to setting signal of the operation control section 32 via the I/F section 231.

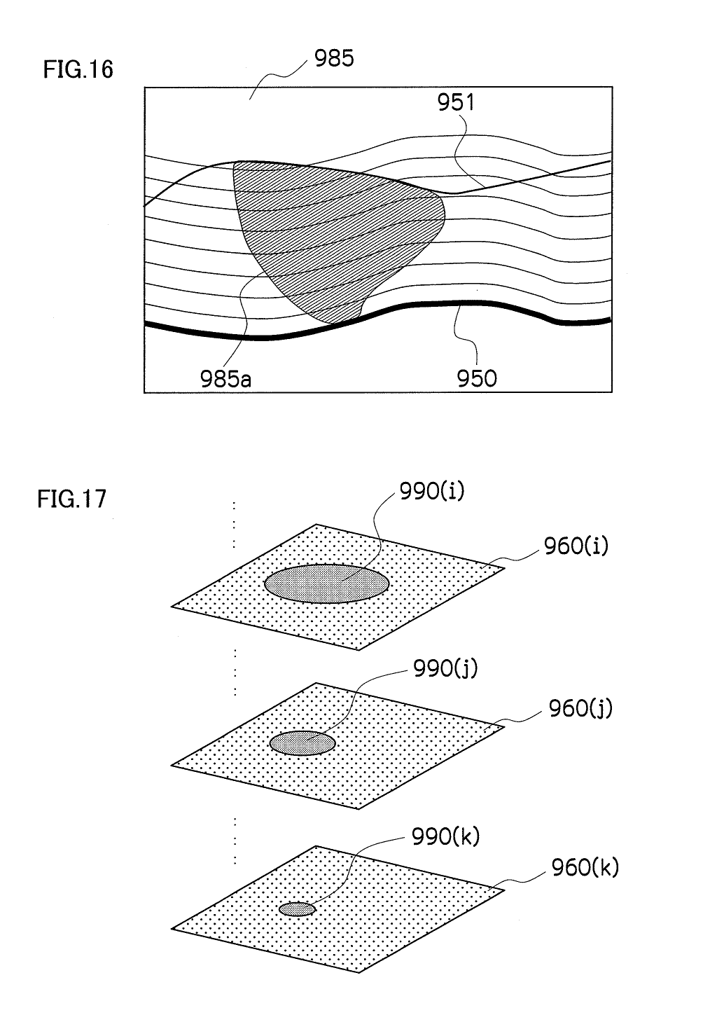

The display control section 230 is capable of displaying the canceration information image 985a as a two-dimensional distribution image of the canceration information on any tomographic image 985 in the depth direction of the stereoscopic characteristic image 982, as shown in FIG. 16, and also of causing the monitor device 500 to display the canceration information images 990 (i), 990 (j) and 990 (k) in the respective parallel regions 960 (i), 960 (j) and 960 (k) of the stereoscopic characteristic images at any of depth positions as the two-dimensional canceration information distribution image, as shown in FIG. 17. The diagnostic imaging apparatus 10 as an optical structure observation apparatus of this embodiment is applicable to any organ in which the crypt structure appears on the mucosal surface, and is applicable to, for example, a stomach, a duodenum, a jejunum, an ileum, a colon and a rectum. This embodiment becomes applicable to an esophagus, a pharynx, a larynx, a bile duct, a pancreatic duct, a bladder, a vagina, a womb and the like characterized by neovascularities appearing on original squamous epitheliums by recognizing the neovascularities. Presence or absence of the pattern peculiar to a neovascularity is recognized using a pattern recognizing method, and by color-coding the regions of a normal site and an abnormal site, thereby enabling the abnormal site in the depth direction to be extracted.

The diagnostic imaging apparatus 10 as the optical structure observation apparatus of the present invention has been described in detail above, but it goes without saying that the present invention is not limited to the above example, and various improvements and modifications may be made within the scope without departing from the gist of the present invention.

What is claimed is:

1. An optical structure observation apparatus which acquires a plurality of pieces of optical structure information of a measured object having a layer structure obtained by scanning a scan surface including a first direction which is a depth direction of the measured object and a second direction orthogonal to the first direction, using a low coherence light, while shifting a position along a third direction which is a direction orthogonal to the scan surface, and constructs an optical stereoscopic structure image based on the acquired plurality of pieces of the optical structure information, comprising:
a calculation region setting device which sets a plurality of calculation regions in the optical stereoscopic structure image;
a region characteristic information calculating device which performs prescribed processing on each calculation region, and calculates region characteristic information of the optical structure information;
a characteristic amount extracting device which extracts a characteristic amount in the calculation region based on the region characteristic information; and
a stereoscopic characteristic image generating device which generates a stereoscopic characteristic image based on the characteristic amount.

2. The optical structure observation apparatus according to claim 1, wherein the calculation region setting device comprises: a middle layer extracting device which extracts a desired middle layer in the measured object from the optical structure information configuring the optical stereoscopic structure image; a layer flattening device which flattens the middle layer; a structure image converting device which reconstructs the optical stereoscopic structure image with the flattened middle layer as a reference layer, and generates a three-dimensional converted optical structure image; and a parallel region setting device which sections the three-dimensional converted optical structure image by a parallel plane parallel to the reference layer on the three-dimensional converted optical structure image, and sets a plurality of parallel regions at prescribed intervals which is orthogonal to the reference layer, the parallel regions being set as the calculation regions.

3. The optical structure observation apparatus according to claim 2, wherein the characteristic amount extracting device comprises: a parallel tomographic image generating device which generates a parallel tomographic image based on the region characteristic information in each parallel region; and an image analyzing device which extracts the characteristic amount by performing a spatial frequency analysis on the parallel tomographic image.

4. The optical structure observation apparatus according to claim 2, wherein the region characteristic information extracting device performs any one of integration processing, maximum intensity projection processing, and minimum intensity projection processing on the optical structure information in the parallel region along a direction orthogonal to the reference layer as the prescribed processing, and thereby extracts the region characteristic information.

5. The optical structure observation apparatus according to claim 2, wherein the stereoscopic characteristic image is a distribution image of the characteristic amount.

6. The optical structure observation apparatus according to claim 2, further comprising: a display control device which causes a display device to display at least any one of the optical stereoscopic image, the three-dimensional converted optical structure image and the stereoscopic characteristic image.

7. The optical structure observation apparatus according to claim 1, wherein the measured object is a living luminal organ.

8. The optical structure observation apparatus according to claim 7, wherein the reference layer is muscularis mucosae.

9. The optical structure observation apparatus according to claim 7, wherein the optical structure information is structure information including crypt structure in the living luminal organ.

10. The optical structure observation apparatus according to claim 7, wherein the characteristic amount represents a degree of canceration of the living luminal organ based on the optical structure information.

11. A structure information processing method for an optical structure observation apparatus which acquires a plurality of pieces of optical structure information of a measured object having a layer structure obtained by scanning a scan surface including a first direction which is a depth direction of the measured object and a second direction orthogonal to the first direction, using a low coherence light, while shifting a position along a third direction which is a direction orthogonal to the scan surface, and constructs an optical stereoscopic structure image based on the acquired plurality of pieces of the optical structure information, the structure information processing method comprising the steps of:
a calculation region setting step which sets a plurality of calculation regions in the optical stereoscopic structure image;
a region characteristic information calculating step which performs prescribed processing on each calculation region, and calculates region characteristic information of the optical structure information;
a characteristic amount extracting step which extracts a characteristic amount in the calculation region based on the region characteristic information; and
a stereoscopic characteristic image generating step which generates a stereoscopic characteristic image based on the characteristic amount.

12. An endoscope apparatus, comprising:
the optical structure observation apparatus according to claim 1; and
an endoscope which includes an insertion section to be inserted into a lumen, wherein a probe emitting and receiving the low coherence light is capable of being inserted into the insertion section.
